(19) United States
(12) Patent Application Publication
(10) Pub. No.: US 2023/0330123 A1
(43) Pub. Date: Oct. 19, 2023

(54) A NON-AQUEOUS SUSPENSION OF ANTICANCER AGENT

(71) Applicant: Auxilla Pharmaceuticals and Research LLP, Ahmedabad (IN)

(72) Inventors: Manish Umrethia, Ahmedabad, Gujarat (IN); Akash Patel, Ahmedabad, Gujarat (IN)

(73) Assignee: Auxilla Pharmaceuticals and Research LLP, Ahmedabad (IN)

(21) Appl. No.: 18/020,646

(22) PCT Filed: Aug. 10, 2021
(86) PCT No.: PCT/IB2021/057356

§ 371 Date: Feb. 10, 2023

(30) Foreign Application Priority Data
Aug. 11, 2020 (IN) 202021034475

(51) Int. Cl. A61K 31/706; A61K 9/00; A61K 9/10; A61K 47/10; A61K 47/18; A61K 47/02

(52) U.S. Cl. CPC A61K 31/706; A61K 9/0019; A61K 9/10; A61K 47/10; A61K 47/18; A61K 47/02

(57) ABSTRACT

The present invention provides a non-aqueous suspension of anticancer agent, particularly injectable suspension. The present invention particularly provides a non-aqueous injectable suspension of anticancer agent comprising therapeutically effective amount of water-soluble anticancer agent, with or without stabilizing agent, non-aqueous vehicle and optionally one or more inactive excipients and also provides process for preparing the same.

### FIELD OF THE INVENTION

The present invention is all about non-aqueous injectable suspension of anticancer agent. Particularly the present invention relates to non-aqueous injectable suspension of anticancer agent comprising water soluble anticancer agent, with or without stabilizing agent, non-aqueous vehicle and optionally one or more inactive ingredients. The present invention also relates to process for preparing the non-aqueous suspension of anticancer agent.

### BACKGROUND OF THE INVENTION

Cancer is a generic term for a large group of diseases that can affect any part of the body. Other terms used are malignant tumours and neoplasms. One defining feature of canceris the rapid creation of abnormal cells that grow beyond their usual boundaries, and which can then invade adjoining parts of the body and spread to other organs, the latter process is referred to as metastasizing. Metastases are a major cause of death from cancer. Cancer arises from the transformation of normal cells into tumour cells in a multistage process that generally progresses from a pre-cancerous lesion to a malignant tumour. These changes are the result of the interaction between a person's genetic factors and 3 categories of external agents, including:
physical carcinogens, such as ultraviolet and ionizing radiation;
chemical carcinogens, such as asbestos, components of tobacco smoke, aflatoxin (a food contaminant), and arsenic (a drinking water contaminant); and
biological carcinogens, such as infections from certain viruses, bacteria, or parasites.

There are different type of cancers and examples of cancer classifications by tissue type include:

Carcinoma: This develops in epithelial tissues, such as those in the gastrointestinal tract or mucous membranes. According to the National Cancer Institute, an estimated 80 to 90 percent of cancer cases are carcinomas.
Leukemia: This is a cancer that arises in the bone marrow, which produces blood cells.
Lymphoma: This develops in the lymphatic system that includes the spleen, tonsils, and thymus. This system relates to immune activity and hormones.
Mixed types: Mixed cancers develop in two different types of cell from one category or multiple categories.
Myeloma: Often occurring in the bone marrow, this type originates in plasma cells that circulate as part of the blood.
Sarcoma: These originate in connective tissue, developing in areas such as the bones, muscle, fat, and cartilage. Sarcomas are more common in young people.

Like different types of cancer, there are different approaches for the treatment is available according to type of cancer and stage of it:
Chemotherapy aims to kill cancerous cells with medications that target rapidly dividingcells. The drugs can also help shrink tumors, but the side effects can be severe.
Hormone therapy involves taking medications that change how certain hormones work or interfere with the body's ability to produce them. When hormones play a significant role, as with prostate and breast cancers, this is a common approach.
Immunotherapy uses medications and other treatments to boost the immune system and encourage it to fight cancerous cells. Two examples of these treatments are checkpoint inhibitors and adoptive cell transfer.
Precision medicine, or personalized medicine, is a newer, developing approach. It involves using genetic testing to determine the best treatments for a person's particular presentation of cancer. Researchers have yet to show that it can effectively treat all typesof cancer, however.
Radiation therapy uses high-dose radiation to kill cancerous cells. Also, a doctor may recommend using radiation to shrink a tumor before surgery or reduce tumor-related symptoms.
Stem cell transplant can be especially beneficial for people with blood-related cancers, such as leukemia or lymphoma. It involves removing cells, such as red or white blood cells, that chemotherapy or radiation has destroyed. Lab technicians then strengthen the cells and put them back into the body.
Surgery is often a part of a treatment plan when a person has a cancerous tumor. Also, a surgeon may remove lymph nodes to reduce or prevent the disease's spread.
Targeted therapies perform functions within cancerous cells to prevent them from multiplying. They can also boost the immune system. Two examples of these therapies are small-molecule drugs and monoclonal antibodies.

From the treatment therapy which includes small molecule drugs or monoclonal antibodies majority of them are in injection dosage form. Among injection dosage form also majority are in lyophilized form where drug is in the form of lyophilized powder which is to be reconstituted at the time of administration.

Lyophilization or freeze drying is a process in which water is removed from a product after it is frozen and placed under a vacuum, allowing the ice to change directly from solid to vapor without passing through a liquid phase. The process consists of three separate, unique, and interdependent processes; freezing, primary drying (sublimation), and secondary drying (desorption).

Lyophilization products have many disadvantages as mentioned below;
Increased handling and processing time
Need for sterile diluent upon reconstitution
Cost and complexity of equipment

Because of their high reactivity in aqueous solutions, many molecules or drugs are difficult to formulate as pharmaceuticals and are often supplied for administration in a lyophilized form that requires reconstitution, usually in water, by skilled hospital personal prior to administration.

Once in aqueous solution, drugs are subject to degradation by hydrolysis, thus, the reconstituted product should be administered to a patient as soon as possible after its reconstitution.

Decitabine is a type of drug called a hypomethylating agent. It works by switching off aprotein called DNA methyltransferase. Decitabine was first approved in USA in 2006 and since then marketed as DACOGEN® in the form of Decitabine Injection which is a sterile, white to off white lyophilized powder/cake, supplied in a clear, colorless glass vial. Each 20 mL, single dose, glass vial contains 50 mg decitabine, 68 mg monobasicpotassium phosphate (potassium dihydrogen phosphate) and 11.6 mg sodium hydroxide.

As per prescribing information, there has been special instruction has been given for the reconstitution of the injection. Further there are three different method has been given for reconstitution based on time of using the injection after reconstitution

Azacitidine for injection contains azacitidine, which is a pyrimidine nucleoside analog of cytidine. Azacitidine is 4-amino-1-β-D-ribofuranosyl-s-triazin-2(1H)-one. The finished product is supplied in a sterile form for reconstitution as a suspension for subcutaneous injection or reconstitution as a solution with further dilution for intravenous infusion. Vials of Azacitidine for injection contain 100 mg of azacitidine and 100 mg mannitol as a sterile lyophilized powder. Cyclophosphamide is a synthetic antineoplastic drug chemically related to the nitrogen mustards. The chemical name for cyclophosphamide is 2-[bis (2-chloroethyl) amino] tetrahydro-2H-1, 3, 2-oxazaphosphorine 2-oxide monohydrate. Cyclophosphamide for Injection, USP is a sterile white powder available as 500 mg, 1 g, and 2 g strength vials.

The main disadvantage of lyophilized drugs is that they have to be reconstituted, usuallyby injecting a diluent through the septum into the vial. The drug is then drawn up into anew syringe, the needle has to be changed before the drug is finally being injected into the patient. These multiple steps are inconvenient and bear the risk of injuries from the exposed needles. This is especially disadvantageous for cytotoxic drugs such as Decitabine.

U.S. Pat. No. 6,982,253 discloses a liquid pharmaceutical composition comprising Decitabine solvated in a solvent that comprises glycerin, propylene glycol, polyethylene glycol, or combinations thereof that comprises less than 40% water.

The pharmaceutical composition further comprises additional excipients e,g. diluents, an acidifying agent and a cyclodextrin. Further the pharmaceutical compositions according to US′253 are unstable and require micronization of the drug due to its limited solubility in the described solvent systems as well as the higher viscosity of the latter.

WO2017162103A1 discloses a preparation method for an azacitidine injection, wherein each azacitidine injection comprises 100 mg of azacitidine and 100 mg of mannitol. The preparation method comprises the steps of measuring 80% water for injection, and controlling the temperature at 2-6° C.; adding mannitol, stirring until completely dissolved, and controlling the temperature at 2-6° C.; adding azacitidine, stirring until completely dissolved, and controlling the temperature at 2-6° C.; sub-packaging into 23 ml/bottle after filtration; freeze-drying according to a preset temperature; and tamponing, capping and packaging after nitrogen filling.

WO2019224756A1 describes stable liquid pharmaceutical composition of decitabine comprising decitabine and at least one pharmaceutically acceptable excipient, wherein moisture content of the liquid composition is less than about 2.0% by weight. The said composition is prepared by using eutectic solvent mixture and antioxidant.

Therefore, there is a need to develop a dosage form or formulations which would be injection-ready to enable delivery of a variety of anticancer agents. There is a further need to develop a non-aqueous injectable suspension for anticancer drugs to offer stable and effective dosage form which can be dispenses easily without much complex process and also to be reconstituted easily without need of strict and technical procedure.

However there is no any stable liquid composition is available which is stable, easy to manufacture and easy to handle or administer and which is ready to use or ready to just dilute before actual administration.

In summary there is still exist a need to have a ready to use and ready to dilute or ready to use non-aqueous suspension of water-soluble anticancer agent which overcomes all problems mentioned in prior art.

The present inventors have arrived at the non-aqueous injectable suspension of anticancer agent to overcome problems cited in above prior arts by preparing a non-aqueous injectable suspension of anticancer agent comprising water soluble anticancer agent, one or more stabilizing agent, non-aqueous injectable vehicle and optionally one or more inactive ingredients as described herein.

### OBJECTIVES OF THE INVENTION

First objective of the present invention is to design and develop a non-aqueous injectable suspension of water soluble anticancer agent.

Another objective of the present invention is to provide a non-aqueous injectable suspension of water-soluble anticancer agent which is ready to use and ready to dilute.

Yet another object of the present invention is to provide non-aqueous injectable suspension of water soluble anticancer agent for the treatment of different types of cancer.

Yet another objective of this invention is to provide an alternative dosage form to existing lyophilized injection of anticancer drugs.

Yet another objective of the present invention is to provide a non-aqueous injectable suspension of water soluble anticancer agent which exhibits bio equivalency against marketed formulations.

More especially, the objective of the present invention is to develop a process for preparing the non-aqueous injectable suspension of water soluble anticancer agent.

### SUMMARY OF THE INVENTION

As per main aspect of the present invention, it provides non-aqueous injectable suspension of anticancer agent comprising water soluble anticancer agent, with or without stabilizing agent, non-aqueous vehicle and optionally one or more inactive ingredients.

In another aspect the present invention is to provide a non-aqueous injectable suspension of watersoluble anticancer agent, which is stable at room temperature.

In another aspect the present invention provides a process for the preparation of a non-aqueous suspension of water soluble anticancer agent.

The details of one or more embodiments of the invention are set forth in the description below. Other features, objects and advantages of the invention will be apparent from the description.

### DETAILED DESCRIPTION OF THE INVENTION

The present invention overcomes the aforesaid drawbacks of the above, and other objects, features and advantages of the present invention will now be described in greater detail. Also, the following description includes various specific details and are to be regarded as merely exemplary. Accordingly, those of ordinary skill in the art will recognize that: without departing from the scope and spirit of the present disclosure and its various embodiments there may be any number of changes and modifications described herein.

The main embodiment of the present invention is to provide a non-aqueous injectable suspension of anticancer agent comprising water soluble anticancer agent, with or without stabilizing agent, non-aqueous vehicle and optionally one or more inactive ingredients.

The non-aqueous injectable suspension of the present invention represents an ideal dosage form to get freedom from complex reconstitution procedure.

The non-aqueous injectable suspension of present invention possesses certain advantages over the other known dosage form of water soluble anticancer agent. It has also been observed that the non-aqueous injectable suspension of water soluble anticancer agent of present invention is more stable.

The non-aqueous injectable suspension of present invention as described herein provide ready to use or ready to dilute dosage form. Various embodiments describe ready to use, liquid composition form in the form of non-aqueous injectable suspension for parenteral route comprising water soluble anticancer drug.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art.

Nothing in this disclosure is to be construed as an admission that the embodiments described in this disclosure are not entitled to antedate such disclosure by virtue of prior invention. As used in this document, the term “comprising” means “including, but not limited to.” “Optional” or “optionally′ means that the subsequently described event or circumstance may or may not occur, and that the description includes instances where the event occurs and instances where it does not.

As used herein, “anticancer agent”, “anti-neoplastic agent” or “anti-tumor agent” generally refers to any agent used in the treatment of cancer. Such agents can be used alone or in combination with other compounds and may alleviate, reduce, ameliorate, prevent, or stand or maintain in remission of clinical symptoms or diagnostic markers associated with neoplasia, tumor or cancer. It can be selected from list including anti-angiogenic agents, alkylating agents, antimetabolites, microtubulin polymerization perturbers (eg, taxol), certain natural products, platinum coordination complexes, anthracenediones, substituted ureas, Methyl hydrazine derivatives, adrenal cortex inhibitors, certain hormones and antagonists, anti-cancer polysaccharides and certain herbs or other plant extracts.

As per preferred embodiment, the anticancer agent of present invention is the drug or active agent which is water soluble.

Solubility of the drug or therapeutic agents in water or an aqueous solvent may vary from being soluble (parts of solvent required for 1 part of solute being 10 to 30), freely soluble(parts of solvent required for 1 part of solute being 1 to 10). As per a preferred embodiment, the anticancer drug of the present invention may be a soluble or freely soluble in water.

As per one embodiment, the anticancer agent of present invention is selected from the group consisting of decitabine, azacytidine, cyclophosphamide, Melphalan, doxorubicin HCl, epirubicin, cytarabine, bortezomib, mitomycin, Bendamustine, vincristine, vinblastine or pharmaceutically acceptable salts thereof.

As per one embodiment, the anticancer agent of present invention is Azacitidine.

As per one embodiment, the anticancer agent of present invention is Decitabine.

As used herein, “cancer” refers to more than 100 diseases caused by uncontrolled proliferation and expansion of abnormal cells that can take the form of non-solid cancers such as solid tumors, lymphomas, and leukemias.

As used herein, “cancer treatment”, “anti-neoplastic treatment”, “cancer therapy”, is defined by reducing the number or proliferation of cancer cells in the body. Although not usually, it refers to any effort to ameliorate or slow the progression of neoplasia, tumor, or cancer by stopping or stopping the growth and division of cancer cells.

As used herein the term “ready to use” or “ready to dilute” means the suspension is either to be used directly for administration without any reconstitution or just need to dilute in to the vehicle to adjust the dose before administration.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

As used herein term “formulation” or “composition” or “dosage” conveys the same meaning and can be used interchangeably.

As used herein, the term “about” means plus or minus 10% of the numerical value of the number with which it is being used.

“Administering” when used in conjunction with a therapeutic means to administer a therapeutic directly into or onto a target tissue or to administer a therapeutic to a patient whereby the therapeutic positively impacts the tissue to which it is targeted.

The term “subject” as used herein includes, but is not limited to, humans and on-human vertebrates such as wild, domestic, and farm animals. In certain embodiments, the subject described herein is an animal. In certain embodiments, the subject is a mammal. In certain embodiments, the subject is a human. In certain embodiments, the subject is a non-human animal. In certain embodiments, the subject is a non-human mammal.

By “pharmaceutically acceptable”, it is meant the carrier, diluent or excipient must be compatible with the other ingredients of the topical formulation and not deleterious to the recipient thereof.

As used herein, the term “therapeutic” means an agent utilized to treat, combat, inhibit, ameliorate, prevent or improve an unwanted condition or disease of a patient.

A ′therapeutically effective amount” or “effective amount” of a composition is a predetermined amount calculated to achieve the desired effect, i.e., to induce a favourable therapeutic response.

The water soluble anticancer agent can be present in the suspension of composition of present invention in an amount from about 1 to 500 mg/mL, preferably in the range from about 1 to about 400 mg/mL, preferably in the range from about 1 to about 300 mg/mL, more preferably in the range from about 1 to 200 mg/mL, about to 500 mg/mL, preferably in the range from about 10 to about 400 mg/mL, preferably in the range from about 10 to about 300 mg/mL, more preferably in the range from about 10 to 200 mg/mL, about 25 to 500 mg/mL, preferably in the range from about 25 to about 400 mg/mL, preferably in the range from about 25 to about 300 mg/mL, more preferably in the range from about 25 to 200 mg/mL or any other range in between thereof.

The term “Suspension” as used herein means a liquid with solid particles dispersed substantially throughout the vehicle. The properties of a liquid suspension, according to the invention, are greatly influenced by the particle size of the anticancer agent. As used herein, a “particle” may be a crystal, a granule, agglomerate, or any un-dissolved solid material. To achieve the rapid onset of activity, which is desirable, a small particle size is essential, ensuring the fastest possible dissolution of the anticancer agent. The particle size distribution in suspension is also a very important factor characterizing the physical stability (for example, sedimentation ratio, etc.) of the formulation. Generally, as the particle size becomes smaller, the sedimentation ratio increases, and the physical stability is improved.

As per one embodiment of present invention, the particle size distribution (PSD) of anticancer agent, that is suitable for suspension according to the invention, includes at least 10% of the particles that are smaller than 30 micron, at least 50% of the particles that are smaller than 70 micron and at least 90% of the particles that are smaller than 200 micron.

As per one preferred embodiment of present invention, the particle size distribution (PSD) of Decitabine is, that is suitable for suspension according to the invention, includes at least 10% of the particles that are smaller than 30 micron, at least 50% of the particles that are smaller than 70 micron and at least 90% of the particles that are smaller than 150 micron.

As per one preferred embodiment of present invention, the particle size distribution (PSD) of Azacitidine is, that is suitable for suspension according to the invention, includes at least 10% of the particles that are smaller than 20 micron, at least 50% of the particles that are smaller than 50 micron and at least 90% of the particles that are smaller than 100 micron.

As per one embodiment of the present invention, the non-aqueous injectable suspension is water free, preferably having water content less than 0.5%.

The stabilizing agent act as the main and critical ingredient to the formulation of present invention. The stabilizing agent make the formulation stable and effective. The stabilizing agent also helps the suspension of present invention more soluble when diluted and at the same time make it stable without increase in degradation.

The stabilizing agent as used herein can be selected from acidifying agent or basifying agent.

Basifying agent as used herein can be selected from trimethylamine, tromethamine, sodium hydroxide, triethanolamine, potassium hydroxide, potassium sorbate, magnesium oxide, calcium carbonate, sodium bicarbonate, meglumine, L-arginine, L-methionine or aluminum hydroxide.

Basifying agent can be present in the non-aqueous injectable suspension of present invention in an amount from about 0.0001 to 200 mg/mL, preferably in the range from about 0.001 to about 200 mg/mL, preferably in the range from about 0.01 to about 200 mg/mL or any other range in between thereof.

Acidifying agent for present invention can be selected from acetic acid, citric acid, fumaric acid, lactic acid, malic acid, phosphoric acid, Potassium dihydrogen phosphate, sodium dihydrogen phosphate, hydrochloric acid and tartaric acid.

Acidifying agent can be present in the non-aqueous injectable suspension of present invention in an amount from about 0.0001 to 200 mg/mL, preferably in the range from about 0.001 to about 200 mg/mL, preferably in the range from about 0.01 to about 200 mg/mL or any other range in between thereof.

As per one embodiment, the non-aqueous injectable suspension of present invention is without any stabilizing agent.

As per one embodiment, the non-aqueous injectable suspension of present invention is comprising only one stabilizing agent.

As per one embodiment, the non-aqueous injectable suspension of present invention is comprising more than one stabilizing agent.

As per one embodiment, the non-aqueous injectable suspension of present invention is comprising only one stabilizing agent and is basifying agent.

As per one embodiment, the non-aqueous injectable suspension of present invention is comprising only one stabilizing agent and is acidifying agent.

As per one embodiment, the non-aqueous injectable suspension of present invention is comprising combination of acidifying agent and basifying agent.

Vehicle for present invention can be used as a base for present invention. Vehicle can be considered as any inert substance, or mixture of substances, added to increase the volume of the liquid composition of present invention in order to make the liquid pharmaceutical composition of the present invention suitable form. It also plays multiple roles in term of also act as solubilizer and to facilitate the solubilization and avoid precipitation.

As per present invention, the vehicle can be selected from aromatic alcohols, lower alkylesters of aryl acids, lower aralkyl esters of aryl acids, aryl ketones, aralkyl ketones, loweralkyl ketones, and lower alkyl esters of citric acid, and combinations thereof.

In a preferred embodiment, the vehicle is ethyl oleate, benzyl benzoate, ethyl benzoate, lauryl lactate, benzyl alcohol, lauryl alcohol, glycofurol, ethanol, tocopherol, polyethylene glycol, glycerol, triacetin, a triglyceride, an alkyltriglyceride, a diglyceride, sesame oil, peanut oil, castor oil, olive oil, cottonseed oil, perfluorocarbon, N-methyl-pyrrolidone, DMSO, glycerol, oleic acid, glycofurol, lauryl lactate, perfluorocarbon, propylene carbonate, or mixtures thereof.

In a more preferred embodiment the vehicle is selected from Propylene glycol, polyethylene glycol, glycerine, medium chain triglycerides.

As per one embodiment, the one or more inactive excipients can be selected from anti-oxidant, preservatives, chelating agents or combinations thereof will be well-known to those of skill in the art.

Anti-oxidants are routinely included in pharmaceutical formulations in order to minimize the oxidative degradation of the drug or active ingredient. The effectiveness of the antioxidants being considered is often assessed by creating several formulations with the API(s) and single or multiple antioxidants in order to place each of these formulations on stability under accelerated conditions.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnamic acid, flavonoids, and their salts, esters, and derivatives). In some embodiments, the anti-oxidant comprises ascorbicacid (vitamin C) and its salts and esters; tocopherols (such as α-tocopherol) and tocotrienols (vitamin E), and their salts and esters.

Preservatives for present invention can included to prevent the growth of microorganisms during the product manufacturing and shelf life. Preservatives can be selected from but not limited to benzoic acid, sodium benzoate, chlorobutanol, ethanol, butyl paraben, propyl paraben and methyl paraben.

Other known pharmaceutical excipients may be used in the ordinary amounts for their normal purposes, so long as they do not negatively affect the effectiveness or stability of the solution.

As per one embodiment, the present invention relates to non-aqueous injectable suspension of anticancer agent for parenteral administration after suitable dilution. Particularly, the liquid composition of present invention is to be administered intravenous route.

As per embodiments, the non-aqueous injectable suspension is sterilized. In some embodiments, the sterilization can be done by any known methods in the art and can be selected from heating, filtration, moist heat sterilization, dry heat sterilization, irradiation, bulk steam sterilization or combination thereof.

As per one embodiment, the present invention relates to process for preparing a non-aqueous suspension of anticancer agent. Particularly, a process for the preparation of a non-aqueous injectable suspension of anticancer agent which is soluble in water, one or more stabilizing agent, non-aqueous vehicle and optionally one or more inactive excipients.

In one embodiment, non-aqueous injectable suspension of present invention is useful for the treatment of cancer disease.

The non-aqueous injectable suspension of the present invention may be used in therapeutic response to anti-cancer therapy disease, disorder or condition in the treatment, prevention of a disease, disorder or condition, ameliorating the disease, disorder or condition, reduce the disease, disorder appearance or condition, or the risk of the disease, disorder or condition appear lower.

As per one embodiment, the present invention provides a process for preparing non aqueous injectable suspension of anti-cancer agent comprising the steps,
a. Adding the non-aqueous vehicle in manufacturing vessel;
b. Adding stabilizing agent in step a) with mixing till dissolving or forming homogenous dispersion;
c. Adding anticancer drug in step b) with mixing and forming homogenous dispersion.

As per one embodiment, the present invention provides a non-aqueous injectable suspension of anticancer agent comprising Decitabine, with or without stabilizing agent, non-aqueous vehicle and optionally one or more inactive ingredients.

As per one embodiment, the present invention provides a Non Aqueous injectable Suspension of Anti-Cancer Agent comprising Decitabine in range from 1 to 500 mg/ml, Triethanolamine and Potassium dihydrogen phosphate in the range from 0.01 to 50 mg/ml, Propylene glycol and polyethylene glycol in the range from 0.1 to 15 ml and one or more inactive agents.

As per one embodiment, the present invention provides a non-aqueous injectable suspension of anticancer agent comprising Azacitidine, one or more stabilizing agent, non-aqueous vehicle and optionally one or more inactive ingredients.

As per one embodiment, the present invention provides non-aqueous injectable suspension of anticancer agent as claimed in claim 16, wherein the Azacitidine is in range from 1 to 500 mg/ml, 0 to 50 ml of Triethanolamine, Potassium dihydrogen phosphate is in the range from 0.01 to 50 mg/ml, Propylene glycol and polyethylene glycol is in the range from 0.1 to 15 ml and one or more inactive agents.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### Examples

### Example 1: Decitabine Injectable Suspension

| | Trial | Trial | Trial | Trial | Trial | Trial | Trial | Trial |
|---|---|---|---|---|---|---|---|---|
| Ingredients | 1.1 | 1.2 | 1.3 | 1.4 | 1.5 | 1.6 | 1.7 | 1.8 |
| Decitabine (mg) | 50 | 50 | 50 | 50 | 50 | 50 | 50 | 50 |
| Propylene | q.s to | — | — | — | — | — | — | — |
| glycol | 0.5 ml | | | | | | | |
| Triethanolamine | — | — | — | 0.04 | 0.4 | 0.75 | 0.75 | 0.75 |
| (mg) | | | | | | | | |
| Potassium | — | — | — | — | — | — | 20 | 30 |
| Dihydrogen | | | | | | | | |
| phosphate (mg) | | | | | | | | |
| Polyethylene | — | q.s to | q.s to | q.s to | q.s to | q.s to | q.s to | q.s to1 |
| glycol | | 0.5 ml | 1 ml | 1 ml | 1 ml | 1 ml | 1 ml | ml |

Process:
1. Propylene glycol was added in manufacturing vessel
2. Triethanolamine was added in step 1) with mixing till dissolved or homogenous dispersion is formed
3. Sterile or non-sterile Decitabine was added in step 2) with mixing tillhomogenous dispersion is formed.

### Example 2: Azacitidine Injectable Suspension

| | Trial | Trial | Trial | Trial | Trial | Trial | Trial |
|---|---|---|---|---|---|---|---|
| Ingredients | 2.1 | 2.2 | 2.3 | 2.4 | 2.5 | 2.6 | 2.7 |
| Azacitidine (mg) | 100 | 100 | 100 | 100 | 100 | 100 | 100 |
| Glycerine (mg) | — | 200 | 200 | — | 200 | — | — |
| Triethanolamine | — | — | 0.75 | — | 0.75 | 0.75 | — |
| (mg) | | | | | | | |
| Potassium | — | — | — | — | 25 | — | — |
| dihydrogen | | | | | | | |
| phosphate (mg) | | | | | | | |
| Propylene | q.s to | q.s to | q.s to | — | — | q.s to | q.s to |
| glycol | 1 ml | 1 ml | 1 ml | | | 1 ml | 1 ml |
| Butylhydroxyanisole | — | — | — | — | — | — | 0.4 mg/ml |
| Polyethylene | — | — | — | q.s to | q.s to | — | |
| glycol 400 | | | | 1 ml | 1 ml | | |

Procedure: As per Example 1

### Example 3: Cyclophosphamide injectable Suspension

| Ingredients | Trial 3.1 | Trial3.2 | Trial3.3 | Trial3.4 | Trial3.5 | Trial3.6 |
|---|---|---|---|---|---|---|
| Cyclophosphamide | 500 | 500 | 500 | 500 | 500 | 500 |
| (mg) | | | | | | |
| Glycerine (mg) | — | 200 | 200 | 200 | 200 | 200 |
| Triethanolamine | — | — | 0.05 | 0.05 | 0.75 | 0.75 |
| (mg) | | | | | | |
| Potassium | — | — | — | — | — | 30 |
| dihydrogen | | | | | | |
| Phosphate (mg) | | | | | | |
| Propylene | q.s to | q.s to | q.s to | q.s to | — | — |
| glycol | 2.5 ml | 2.5 ml | 2.5 ml | 5 ml | | |
| Polyethylene | — | — | — | — | q.s to | q.s to |
| glycol | | | | | 5 ml | 5 ml |

Procedure: As per Example 1

### Example 4: Analysis of Suspension Prepared in Example 1

TABLE 1
Comparative stressed stability data of formulations prepared with
different vehicles as per Trial 1.1 and Trial 1.2

| | Trial 1.1 | | Trial 1.2 | |
|---|---|---|---|---|
| | (propylene glycol) | | (Polyethylene glycol) | |
| | | 60° C. | | 60° C. |
| Parameters | Initial | 96 hour | Initial | 96 hour |
| % Assay | 97.6% | 89.0% | 99.9% | 93.6% |
| % of 5-Azacytosine | ND | 0.3% | ND | 0.09% |
| impurity | | | | |
| % of α-Anomer | 0.04% | 0.08% | 0.04% | 0.05% |
| impurity | | | | |
| % of Ester impurity | 0.02% | 0.02% | 0.02% | 0.02% |
| % of highest | 0.06% | 6.2% | 0.09% | 3.3% |
| individual | | | | |
| unspecified impurity | | | | |
| % of total impurities | 0.12% | 9.0% | 0.22% | 5.0% |

Conclusion: Based on above data, the polyethylene glycol used in Trial 1.2 is morecompatible than propylene glycol used in Trial 1.1.

Effect of Stabilizing Agent on Formulation

The goal of this study was to evaluate effect of stabilizing agent and below table represents formulations with and without presence of selected stabilizing agent.

TABLE 2
Comparative stress study of formulations with and without
stabilizing agent as per Trial 1.3 and Trial 1.4

| | Trial 1.3 | | Trial 1.4 | |
|---|---|---|---|---|
| | | 60° C. | | 60° C. |
| Parameters | Initial | 96 hour | Initial | 96 hour |
| % Assay | 95.9% | 98.9% | 99.8% | 99.4% |
| % of 5-Azacytosine | ND | 0.06% | ND | ND% |
| impurity | | | | |
| % of α-Anomer | 0.04% | 0.04% | 0.04% | 0.04% |
| impurity | | | | |
| % of Ester impurity | 0.02% | 0.02% | 0.02% | 0.02% |
| % of highest | 0.06% | 1.3% | 0.06% | 0.4% |
| individual | | | | |
| unspecified impurity | | | | |
| % of total impurities | 0.12% | 2.3% | 0.22% | 1.2% |

Conclusion: Based on above data with available analytical method, it is concluded thatTrial 1.4 (presence of triethanolamine) keeps product more stable than that in Trial 1.3.

Optimization of Stabilizing Agent

The goal of this study was to determine optimum concentration of stabilizing agent which can keep formulation more stable after reconstitution. There were three different concentrations of triethanolamine used and diluted with water for injection and tested forrelated substances.

TABLE 3
Optimization of triethanolamine after dilution with water for injection

| Parameters | Trial 1.4 | Trial 1.5 | Trial 1.6 |
|---|---|---|---|
| % Assay | 95.9% | 87.7% | 85.7% |
| % of 5-Azacytosine | ND | ND | ND |
| impurity | | | |
| % of α-Anomerimpurity | 0.04% | 0.04% | 0.04% |
| % of Ester impurity | 0.02% | 0.02% | 0.02% |
| % of highest individual | 0.05% @ | 0.20% @ | 0.65% @ |
| impurity | 0.56 RRT | 0.56 RRT | 0.56 RRT |
| unspecified | 1.9% @ | 10.5% @ | 15.5% @ |
| | 1.23 RRT | 1.23 RRT | 1.23 RRT |
| % of total impurities | 2.0% | 10.8% | 16.2% |

Conclusion: As per above data with available analytical method, it is concluded that impurity @ 1.23 RRT is significantly higher in case of Trial 1.5 and 1.6 due to presence of higher concentration of triethanolamine. Therefore, it can be concluded that there is an optimized concentration of stabilizing agent required to control impurity profile with and without dilution. Based on above data, Trial 1.4 is better formulation compared to Trial 1.5 and 1.6.

Optimization of Stabilizing Agent without Reconstitution

The goal of the study was to evaluate role of concentration of stabilizing agent without dilution to keep formulation stable. To determine the same, three batches were prepared, 1 ml filled in glass vials and studied after 60° C. 96 hr.

TABLE 4
Comparative stress study of formulations with various concentrations
of stabilizing agent without reconstitution with water

| Parameters | Trial 1.4 | | Trial 1.5 | | Trial 1.6 | |
|---|---|---|---|---|---|---|
| % Assay | 99.8% | 99.4% | 101.2% | 102.9% | 98.7% | 100.0% |
| % of 5- | ND | ND | ND | ND | ND | ND |
| Azacytosine | | | | | | |
| impurity | | | | | | |
| % of α- | 0.04% | 0.04% | 0.04% | 0.04% | 0.04% | 0.04% |
| Anomer | | | | | | |
| impurity | | | | | | |
| % of Ester | 0.02% | 0.02% | 0.02% | 0.02% | 0.03% | 0.03% |
| impurity | | | | | | |
| % of | 0.06% | 0.4% | 0.07% | 0.25% | ND | 0.24% |
| highest | | | | | | |
| individual | | | | | | |
| unspecified | | | | | | |
| impurity | | | | | | |
| % of total | 0.22% | 1.2% | 0.18% | 0.47% | 0.07% | 0.65% |
| Impurities | | | | | | |

Conclusion: Based on above data using available method, it is found that as triethanolamine' s concentration increases, % of highest individual unspecified related substances decreases.

Effect of Two Stabilizing Agents

The goal of this study was to optimize formulation composition of Trial 1.6, which is much stable without dilution with water for injection but degrade faster when mixed with water for injection. Therefore, Trial 1.6 was further optimized using second stabilizing agent at 2 different concentrations.

TABLE 5
Comparative impurity profile of two formulations in presence of
second stabilizing agent and against Marketed Product (RLD) after
reconstitution with water for injection.

| | | | Marketed |
|---|---|---|---|
| Parameters | Trial 1.7 | Trial 1.8 | formulation* |
| % of 5-Azacytosine | ND | ND | ND |
| impurity | | | |
| % of α-Anomer | 0.04% | 0.04% | 0.04% |
| impurity | | | |
| % of Ester impurity | 0.02% | 0.02% | 0.02% |
| % of highest | 3.0% @ | 2.0% @ | 2.3% @ |
| individual unspecified | 1.23 RRT | 1.23 RRT | 1.23 RRT |
| impurity | | | |
| % of total impurities | 3.4% | 2.3% | 2.4% |

*Marketed formulation used was DACOGEN ®

Conclusion: As per above data, it is concluded that degradation of Trial 1.6 can be improved by addition of second stabilizing agent. Trial 1.8 is having similar degradation profile as Marketed formulation after dilution with water for injection.

Stability Data of Trial 1.4:

| | | 25° C./60% RH | | | | 40° C./75% RH | | | |
|---|---|---|---|---|---|---|---|---|---|
| | Initial | | | | 6 M | | | | 6 M |
| Parameters | 0 M | 1 M | 3 M | 6 M | reconstitution | 1 M | 3 M | 6 M | reconstitution |
| % Assay | 104.2% | 96.6% | 96.8% | 99.0% | 96.6% | 102.4% | 100.0% | 98.5% | 91.4% |

Related Substances

| | | | | | | | | | |
|---|---|---|---|---|---|---|---|---|---|
| % of 5- | ND | ND | BLQ | BLQ | 0.03% | 0.02% | 0.14% | 0.14% | 0.22% |
| Azacytosine | | | | | | | | | |
| % of α- | 0.04% | 0.04% | 0.04% | 0.04% | 0.05% | 0.04% | 0.04% | 0.04% | 0.05% |
| Anomer | | | | | | | | | |
| % of Ester | 0.02% | 0.02% | 0.04% | 0.02% | 0.02% | 0.02% | 0.03% | 0.02% | 0.02% |
| impurity | | | | | | | | | |
| % of highest | BLQ | BLQ | 0.13% | 0.05% | 1.6% | 0.2% | 0.56% | 0.65% | 1.0% |
| individual | | | | | | | | | |
| unspecified | | | | | | | | | |
| impurity | | | | | | | | | |
| % of total | 0.07% | 0.06% | 0.65%^{#} | 0.16% | 3.70% | 0.40% | 2.98% | 2.35% | 5.69% |
| impurities | | | | | | | | | |

| | |
|---|---|
| Water content | 0.15% |
| Particle size | D(10)-17.1 μm |
| distribution^{@} | D(50)-54.9 μm |
| | D(90)-125 μm |

### Example 5: Suitability and Stability of Azacitidine in Various Vehicles

The goal of this study was to assess suitability of Azacitidine in various vehicles. The below listed excipients/vehicles were tried to suspend azacitidine. For each experiment, 100 mg of Azacitidine was taken in a beaker, respective vehicle was added slowly using micropipette and exposed to stressed condition to check compatibility of azacitidine.

| | B. No. | | | | | |
|---|---|---|---|---|---|---|
| | Trial 2.4 | | Trial 2.1 | | Trial 2.4 | |
| | Composition | | | | | |
| | Azacitidine + | | Azacitidine + | | | |
| | Polyethylene | | Propylene | | Azacitidine + | |
| | glycol 400 | | glycol | | Glycerine | |
| | Condition | | | | | |
| | | 60° C. | | 60° C. | | 60° C. |
| | Initial | 48 hrs | Initial | 48 hrs | Initial | 48 hrs |
| % Assay | 103.2 | 103.7 | 102.8 | 100.4 | 102.5 | 91.1 |
| % Related compound A | BQL | BQL | BQL | BQL | BQL | BQL |
| % Acetylated Impurity | ND | ND | ND | ND | ND | ND |
| % Highest unspecified | BQL | BQL | BQL | BQL | BQL | BQL |
| impurity | | | | | | |
| % Related Compound C | 0.09 | 0.09 | 0.18 | 2.29 | 0.19 | 4.89 |
| % Total Impurities | 0.09 | 0.09 | 0.18 | 2.29 | 0.19 | 5.10 |

Conclusion: Based on above experiments, it was observed that there is increase in Azacitidine related compound C in case of Propylene glycol and Glycerin. There is no change found in assay and related substances in case of Polyethylene glycol 400. Further short term stability with Polyethylene glycol 400 were performed.

Thermal Stability with Polyethylene Glycol 400

Short term stability study with Polyethylene glycol 400 were performed

| | B. No. | | | |
|---|---|---|---|---|
| | AZAP/150920/1002 | | | |
| | Composition | | | |
| | Azacitidine + polyethylene glycol 400 | | | |
| | | 40° C./75% RH | | 25° C./65% RH |
| Condition | Initial | 1M | 3M | 3M |
| % Assay | 100.0 | 99.8 | 97.1 | 96.9 |
| % Related compound A | BQL | BQL | BQL | BQL |
| % Acetylated Impurity | ND | ND | ND | ND |
| % Highest unspecified | BQL | BQL | BQL | BQL |
| impurity | | | | |
| % Related Compound C | 0.31 | 0.61 | 0.61 | 0.39 |
| % Total Impurities | 0.31 | 0.61 | 0.67 | 0.68 |

Conclusion: Based on above data using available method, it is found that Azacitidine with polyethylene glycol is stable up to 3M 40° C./75% RH. All impurities levels are well within acceptance criteria.

Reconstitution and Dilution Stability Study

Goal of this study was to check the stability of reconstituted and diluted product of Azacitidine and to compare it with in-house prepared RLD composition.

Conclusion: Based on below data, it can be concluded that the impurity profile of Test product is comparable to that of in-house prepared RLD composition. Two impurities (Azacitidine related compound C & Formyl amidine analogue) were found above identification threshold in test product as well as RLD.

Stability Data of Reconstituted Product (Trial 2.4)

| | B. No. | | | | | |
|---|---|---|---|---|---|---|
| | AZAP/030221/1010A/B/C | | | AZAP/030221/1010D/E/F | | |
| | Composition | | | | | |
| | | | | RLD composition (In-house | | |
| | Test Product | | | preparation) | | |
| | Condition | | | | | |
| | | Reconstitution | Reconstitution | | Reconstitution | Reconstitution |
| | Dilution^{@} | (25 mg/mL) | (25 mg/mL) | Dilution | (25 mg/mL) | (25 mg/mL) |
| | Immediate | 25° C. | 5° C. | Immediate | 25° C. | 5° C. |
| | (1 mg/mL) | 1 hour | 22 hrs | (1 mg/mL) | 1 hr | 22 hrs |
| % Assay | 97.6 | 97.5 | 99.6 | 87.5 | 94.1 | 90.2 |
| % Related | 0.1 | 0.07 | 0.07 | 0.07 | 0.07 | 0.079 |
| compound A | | | | | | |
| % Acetylated | ND | ND | ND | ND | ND | ND |
| Impurity | | | | | | |
| % Highest | ND | ND | 0.06 | 0.10 | 0.09 | 0.11 |
| unspecified | | | | | | |
| impurity | | | | | | |
| % Related | 0.41 | 0.40 | 0.59 | 0.56 | 0.55 | 0.99 |
| Compound C | | | | | | |
| Formyl | 6.0 | 1.50 | 2.50 | 8.70 | 2.90 | 3.00 |
| amidine | | | | | | |
| analog | | | | | | |
| Total Imp^{#} | 0.51 | 0.47 | 0.72 | 0.72 | 0.72 | 1.20 |

Reconstitution and Dilution Stability Study

| | Decitabine for Injection | | | |
|---|---|---|---|---|
| Parameter | Reference product | | Trial 1.4 | |
| % Assay | 97.8% | 94.0% | 98.5% | 95.2% |

Related Substances

| | | | | |
|---|---|---|---|---|
| % of | ND | ND | BLQ | ND |
| 5-Azacytosine | | | | |
| impurity | | | | |
| % of α-Anomer | ND | ND | 0.092% | 0.094% |
| impurity | | | | |
| % of Ester | 0.024% | 0.033% | 0.033% | 0.039% |
| impurity | | | | |
| % of highest | 2.0% | 1.7% | 1.2% | 1.1% |
| individual | (RRT 1.26) | (RRT 1.26) | (RRT 1.23) | (RRT 1.23) |
| unspecified | | | | |
| impurity | | | | |
| % of total | 2.02% | 1.73% | 1.33% | 1.23% |
| impurities | | | | |

Conclusion: Based on above data, it can be concluded that the impurity profile of Test product is comparable to that of Decitabine for Injection (Marketed product). Two impurities at RRT 1.26 & RRT 1.87 was found above identification threshold in test product as well as in the marketed product.

Effect of Stabilizer

| | Trial 2.1 | | Trial 2.6 | |
|---|---|---|---|---|
| | Azacitidine + PG | | Azacitidine + Triethanolamine + PG | |
| | Initial | 121° C. 15 min | Initial | 121° C. 15 min |
| RRT | % Impurities | | % Impurities | |
| Azacitidine | BQL | 0.26 | BQL | 0.21 |
| compound | | | | |
| A | | | | |
| 0.35 RRT | ND | 0.1 | ND | 0.07 |
| 0.40 RRT | ND | 0.26 | ND | 0.17 |
| 0.44 RRT | ND | 0.33 | ND | 0.29 |
| 0.50 RRT | ND | 0.34 | ND | 0.37 |
| 0.52 RRT | BQL | 0.6 | BQL | 0.62 |
| 0.56 RRT | ND | 0.09 | ND | 0.07 |
| 0.66 RRT | ND | 0.5 | ND | 0.35 |
| 0.81 RRT | ND | 0.13 | ND | 0.18 |
| Total % | ND | 2.61 | ND | 2.33 |

Conclusion: Based on above data, it was concluded that total impurities are increasing in absence of stabilizer (Triethanolamine) in comparison to without stabilizer.

1. A non-aqueous injectable suspension of anticancer agent comprising water soluble anticancer agent, with or without stabilizing agent, non-aqueous vehicle and optionally one or more inactive ingredients.

2. The non-aqueous injectable suspension of anticancer agent as claimed in claim 1, wherein water soluble anticancer agent is selected from decitabine, azacitidine, cyclophosphamide, Melphalan, doxorubicin HCl, epirubicin, cytarabine, bortezomib, mitomycin, Bendamustine, vincristine, vinblastine or pharmaceutically acceptable salts thereof.

3. The non-aqueous injectable suspension of anticancer agent as claimed in claim 1, wherein the anti-cancer agent is present in range from 1 to 500 mg/ml.

4. The non-aqueous injectable suspension of anti-cancer agent as claimed in claim 1, wherein stabilizing agent is selected from trimethylamine, tromethamine, sodium hydroxide, triethanolamine, potassium hydroxide, potassium sorbate, magnesium oxide, calcium carbonate, sodium bicarbonate, meglumine, L-arginine, L-methionine or aluminum hydroxide, acetic acid, citric acid, fumaric acid, lactic acid, malic acid, phosphoric acid, Potassium dihydrogen phosphate, sodium dihydrogen phosphate, hydrochloric acid and tartaric acid and combination thereof.

5. The non-aqueous injectable suspension of anti-cancer agent as claimed in claim 1, wherein the stabilizing agent is present in range from 0.01 to 200 mg.

6. The non-aqueous injectable suspension of anti-cancer agent as claimed in claim 1, non-aqueous vehicle is selected from Propylene glycol, polyethylene glycol, glycerine, medium chain triglycerides.

7. The non-aqueous injectable suspension of anti-cancer agent as claimed in claim 1, wherein non-aqueous vehicle is in range from 200-1000 mg/ml.

8. The non-aqueous injectable suspension of anti-cancer agent as claimed in claim 1, wherein the anticancer drug in range 1 to 500 mg/ml, Triethanolamine and Potassium dihydrogen phosphate is in range 0.01 to 50 mg/ml, Propylene glycol and polyethylene glycol is in range from 0.1 to 15 ml and one or more inactive agents.

9. The process for preparing the non-aqueous injectable suspension of anti-cancer agent as claimed in claim 1 is comprising the steps;
a. Adding the non-aqueous vehicle in manufacturing vessel;
b. Adding stabilizing agent in step a) with mixing till dissolving or forming homogenous dispersion;
c. Adding anticancer drug in step b) with mixing and forming homogenous dispersion.

10. A non-aqueous injectable suspension of anticancer agent comprising Decitabine, with or without stabilizing agent, non-aqueous vehicle and optionally one or more inactive ingredients.

11. The non-aqueous injectable suspension of anticancer agent as claimed in claim 10, wherein Decitabine is present in range from 1 to 500 mg/ml.

12. The non-aqueous injectable suspension of anticancer agent as claimed in claim 10, wherein stabilizing agent is selected from trimethylamine, tromethamine, sodium hydroxide, triethanolamine, potassium hydroxide, potassium sorbate, magnesium oxide, calcium carbonate, sodium bicarbonate, meglumine, L-arginine, L-methionine or aluminum hydroxide, acetic acid, citric acid, fumaric acid, lactic acid, malic acid, phosphoric acid, Potassium dihydrogen phosphate, sodium dihydrogen phosphate, hydrochloric acid and tartaric acid and combination thereof.

13. The non-aqueous injectable suspension of anticancer agent as claimed in claim 10, wherein the stabilizing agent is present in range from 0.01 to 200 mg.

14. The non-aqueous injectable suspension of anticancer agent as claimed in claim 10, non-aqueous vehicle is selected from Propylene glycol, polyethylene glycol, glycerine, medium chain triglycerides.

15. The non-aqueous injectable suspension of anticancer agent as claimed in claim 10, wherein the Decitabine is in range from 1 to 500 mg/ml, Triethanolamine and Potassium dihydrogen phosphate is in the range from 0.01 to 50 mg/ml, Propylene glycol and polyethylene glycol is in the range from 0.1 to 15 ml and one or more inactive agents.

16. A non-aqueous injectable suspension of anticancer agent comprising Azacitidine, one or more stabilizing agent, non-aqueous vehicle and optionally one or more inactive ingredients.

17. The non-aqueous injectable suspension of anticancer agent as claimed in claim 16, wherein Azacitidine is present in range from 1 to 500 mg/ml.

18. The non-aqueous injectable suspension of anticancer agent as claimed in claim 16, wherein one or more stabilizing agent is selected from trimethylamine, tromethamine, sodium hydroxide, triethanolamine, potassium hydroxide, potassium sorbate, magnesium oxide, calcium carbonate, sodium bicarbonate, meglumine, L-arginine, L-methionine or aluminum hydroxide, acetic acid, citric acid, fumaric acid, lactic acid, malic acid, phosphoric acid, Potassium dihydrogen phosphate, sodium dihydrogen phosphate, hydrochloric acid and tartaric acid and combination thereof.

19. The non-aqueous injectable suspension of anticancer agent agent as claimed in claim 16, wherein the stabilizing agent is present in range from 0.01 to 200 mg.

20. The non-aqueous injectable suspension of anticancer agent as claimed in claim 16, non-aqueous vehicle is selected from Propylene glycol, polyethylene glycol, glycerine, medium chain triglycerides.

21. The non-aqueous injectable suspension of anticancer agent as claimed in claim 16, wherein the Azacitidine is in range from 1 to 500 mg/ml, 0 to 50 ml of Triethanolamine, Potassium dihydrogen phosphate is in the range from 0.01 to 50 mg/ml, Propylene glycol and polyethylene glycol is in the range from 0.1 to 15 ml and one or more inactive agents.
